(19) United States
(12) Patent Application Publication
(10) Pub. No.: US 2020/0184503 A1
(43) Pub. Date: Jun. 11, 2020

(54) PHYSICAL ACTIVITY ASSESSMENT SYSTEM AND METHOD TO DETECT FAULTY PHYSICAL ACTIVITY DATA

(71) Applicant: For Long Life Corp, Bridgewater, NJ (US)

(72) Inventors: Shiquan Peng, Bridgewater, NJ (US); Jun Li, Bridgewater, NJ (US)

(21) Appl. No.: 16/789,378

(22) Filed: Feb. 12, 2020

Related U.S. Application Data
(63) Continuation-in-part of application No. 16/141,144, filed on Sep. 25, 2018.
(60) Provisional application No. 62/714,669, filed on Aug. 4, 2018.

(51) Int. Cl. G06Q 30/02; G06N 20/00; G06N 5/04; A61B 5/024; A61B 5/00

(52) U.S. Cl. CPC G06Q 30/0225; G06N 20/00; A61B 5/746; A61B 5/024; A61B 5/6801; G06N 5/04

(57) ABSTRACT

A physical activity assessment system (PAAS), a method, and a computer program product may be provided to detect faulty physical activity (PA) data for claiming a reward. The PAAS may include a memory configured to store computer program code and a processor configured to execute the computer program code to obtain first PA data of a first wearable device associated with a first user. The first wearable device may be linked to a first user device registered with the PAAS by the first user as an owner. The first user may claim for a reward based on the first PA data. The processor may be configured to detect whether the first PA data is faulty PA data generated by the first wearable device worn by a non-owner of the first wearable device, based on fraud detection criteria.

### TECHNOLOGICAL FIELD

The present disclosure generally relates to a physical activity assessment system (PAAS) and more particularly to the PAAS to detect faulty physical activity data generated for a user to claim reward.

### BACKGROUND

Wearable devices for fitness have become popular in recent years. Physical Activity (PA) data generated by a wearable device (such as a fitness band) worn by a person may be used as a proof of exercise for the person to gain a reward from an interested party. The PA data may include, but not limited to, walk steps and heart rate. For example, an organization may want to reward their employees when the employees exercise regularly.

In certain scenarios, a system may be used that automatically rewards for physical activities of people. In such scenarios, the people may have incentive to cheat the system by generation of false data to get a reward with less or no actual effort of performing physical activities. For example, a person may ask other person or a non-human to wear the fitness band to generate the PA data and use the generated PA data to claim a reward. In another exemplary scenario, a person may wear more than one fitness bands and claim multiple rewards for a single physical activity.

In a certain scenario, a system is used to detect the fraud of wearing two or more wearable devices by same person that requires real-time peer-to-peer (P2P) connections between wearable devices. However, the problem is that normally a wearable device is designed only to be able to pair with a smartphone and not between two wearable devices. In certain other scenarios, firmware modifications for wearable devices may be required to improve the compatibility of the wearable device among multiple device manufacturers.

### BRIEF SUMMARY

A system, a method, and a computer program product are provided herein that focuses on detecting faulty physical activity (PA) data for claiming a reward. The disclosed system may work at three levels namely, on wearable devices, user devices (smartphones) and/or distributed servers (blockchain nodes) which relax the requirements of firmware modifications for wearable devices and improving the device compatibility among multiple device manufacturers.

Embodiments disclosed herein may provide a physical activity assessment system (PAAS) to detect faulty physical activity (PA) data for claiming a reward. The PAAS may include a memory configured to store computer-executable instructions and a processor configured to execute the computer-executable instructions to obtain first PA data of a first wearable device associated with a first user. The first wearable device may be linked to a first user device registered with the PAAS. The first user may claim for a reward based on the first PA data. The processor may be configured to detect whether the first PA data is faulty PA data generated by the first wearable device when worn by a non-owner of the first wearable device, based on fraud detection criteria.

In accordance with an embodiment, the fraud detection criteria may comprise at least one of a first criterion or a second criterion. The first criterion includes a first condition that a second user who is the non-owner of the first wearable device wears the first wearable device associated with the first user for a single physical activity. The second criterion includes a second condition that the first user wears both the first wearable device and a second wearable device at a same time for the single physical activity, wherein the second wearable device is not registered by the first user.

In accordance with an embodiment, the PAAS may further comprise a network timer. The processor may be further configured to synchronize a time stamp of a plurality of wearable devices that are registered with the PAAS based on the network timer. The plurality of wearable devices that are registered with the PAAS may comprise the first wearable device and the second wearable device.

In accordance with an embodiment, the first PA data may comprise first PA burst data and first extended PA burst data. The first PA burst data comprises a first heart rate for a first time period. Further, second PA data is generated from the second wearable device for a second time period, and the second PA data comprises second PA burst data and second extended PA burst data. The second PA burst data comprises a second heart rate generated at the second time period which overlaps with the first time period. The processor may be further configured to determine a correlation of the first heart rate in the first PA data of the first wearable device generated for the first time period with the second heart rate of the second PA data of the second wearable device generated at the second time period, based on the first PA burst data, the first extended PA burst data, the second PA burst data and the second extended PA burst data. The correlation may be a high correlation based on value of the correlation being greater than a first threshold value. The processor may be further configured to flag at least one of the first PA data of the first wearable device or the second PA data of the second wearable device generated for the first time period as the faulty PA data, based on the high correlation. The high correlation associates with the first criterion or the second criterion. A correlation period of the correlation between the first PA data and the second PA data corresponds to a time overlap between the first time period and the second time period. The correlation period comprises a finite number of time instances

In accordance with an embodiment, the processor may be further configured to calculate a cross variance between the first PA data of the first wearable device and the second PA data of the second wearable device for determination of the correlation of the first PA data of the first wearable device and the second PA data of the second wearable device.

In accordance with an embodiment, the cross variance is calculated as:

$$Cross variance ( Var 1 , 2 [ K ] ) = \frac{Σ k = 1 … K \{ [ R 1 [ k ] - r 1 ] [ R 2 [ k ] - r 2 ] \}}{( Sqrt [ Var 1 ] * Sqrt [ Var 2 ] )}$$

wherein (Var1,2[K]) is a cross variance between two PA data sets (the first PA data and the second PA data) of K samples at K synchronized time instances, wherein r1 and r2 are means of R1 [k]} and R2[k]} respectively for K samples, and wherein Var1 and Var2 are variances of {R1[k]} and R2[k]} for K samples, respectively.

In accordance with an embodiment, the processor may further be configured to determine missing data on one of the R1[k] or the R2[k] in the K time instances, and fill the missing data by using either interpolation technique or extrapolation technique.

In accordance with an embodiment, the processor may be further configured to control an application on the first wearable device associated with the first PA data and an application on the second wearable device associated with the second PA data to prompt an alarm for violation of terms of use or a usage policy based on the generation of the faulty PA data.

In accordance with an embodiment, the processor may be further configured to determine first and second derivatives of the first PA data of the first wearable device and the second PA data of the second wearable device, respectively. The processor may be further configured to determine the high correlation between the first PA data and the second PA data, based on the first and second derivatives of the first PA data and the second PA data, respectively.

In accordance with an embodiment, the first extended PA burst data and the second extended PA burst data may comprise at least one of a rest time data, ramp up period data, exercise period data, slow-down period data, and recovery period data. The first PA burst may comprise data associated with a physical activity during an exercise period that count towards the claim for reward.

In accordance with an embodiment, the processor may be further configured to control an application on the first user device to raise an alarm for fraud detection, based on a sudden change in the first PA burst data during physical activity period. The sudden change indicates a presence of the missing data in the first PA burst data during an extended time period of the physical activity.

In accordance with an embodiment, the first PA data of the first wearable device associated with the first user further may comprise historical data of the first user who is the owner of the first wearable device. The processor may be further configured to obtain a trained machine learning model trained on the historical data of the first user and detect whether the first PA data is the faulty PA data generated by the first wearable device worn by a non-owner of the first wearable device, based on the trained machine learning model.

In accordance with an embodiment, the historical data of the first user may correspond to biometric data of the first user. The biometric information of the first user may comprise at least one of rest time data, heart rate data, ramp up pattern data, slow down pattern data, or recovery time data.

In accordance with an embodiment, the processor may be further configured to determine second derivative of the first PA data and detect the first PA data being a faulty PA data generated by the first wearable device worn by a non-owner, based on deviation of the second derivative of the first PA data from the historical data of the first PA data.

In accordance with an embodiment, the processor may be further configured to receive a request from the application of the first user device to find a paired application of the second user device, based on the first user device receiving the first PA burst data from the first wearable device with the second wearable device in a vicinity of the first wearable device's signal coverage. The processor may be further configured to identify the paired application of the second user device associated with the second wearable device, based on the first wearable device being in a vicinity of the second wearable device. The processor may be further configured to transmit an application identity number of the second user device to the first user device and control the first user device to transmit fraud detection request to the second user device along with the first PA burst data and the application identity number of the second user device which is in the vicinity of the first user device.

In accordance with an embodiment, the first PA burst data may further comprise identity of family and friends of the first user associated with the first wearable device. The processor may be further configured to detect whether the first PA burst data is faulty PA burst data generated by the first wearable device worn by the non-owner, based on Virtual Friend Group (VFG) criteria.

In accordance with an embodiment, the VFG criteria may comprise at least one of a first criterion that the second user has a common non-public Point of Interest (PoI) with the first user. The non-public PoI may comprise home and office. The home may be assigned more weightage than the office as non-public PoI. A second criterion that a number of people in the non- public PoI are less than a third pre-defined threshold value. A third criterion that the second user has a common non-public PoI and an exercise PoI with the first user. The exercise PoI may correspond to a location where the first PA burst data is generated or a fourth criterion that a network address and a Global Positioning System (GPS) value of the second user is same as that of the first user. Another criterion may correspond to the second burst data from the second user device which is registered in the VFG of the first user.

Embodiments disclosed herein may provide a fraud detection distributed server comprising a plurality of nodes. A first node of the plurality of nodes may be configured to receive a reward claim with a first physical activity (PA) burst data from a first user device paired with a first wearable device associated with a first user. The first PA burst data may comprise an identity of a second wearable device in nearby device field having time overlap with the first PA burst data. The first node of the plurality of nodes may be configured to transmit a fraud detection request message to a second node of the plurality of nodes responsible to process reward claims from a second user device associated with the second wearable device. The first node of the plurality of nodes may be configured to receive a result of correlation from the second node for the first PA burst data of the first user device with a second PA burst data of the second user device and detect fraud for disqualification of the reward claim, based on the result being a high correlation of the first PA burst data with the second PA burst data.

In accordance with an embodiment, the first node of fraud detection distributed server may be further configured to accept qualification of the reward claim of the first user device, based on the result being a non-correlation of the first PA burst data with the second PA burst data.

In accordance with an embodiment, the first node may be responsible for processing reward claim from the first user device. The first user device may be registered with the fraud detection distributed server. The plurality of nodes may correspond to blockchain nodes.

Embodiments disclosed herein may provide a method for obtaining first PA data of a first wearable device associated with a first user. The first wearable device may be linked to a first user device registered with the PAAS. The first user may claim for a reward based on the first PA data and detecting whether the first PA data is faulty PA data generated by the first wearable device when worn by a non-owner of the first wearable device, based on fraud detection criteria.

Embodiments disclosed herein may provide a method for a method for fraud detection distributed server comprising a plurality of nodes. The method may comprise receiving a reward claim with a first physical activity (PA) burst data from a first user device paired with a first wearable device associated with a first user. The first PA burst data may comprise an identity of a second wearable device in nearby device field having time overlap with the first PA burst data. The method may comprise transmitting a fraud detection request message to a second node of the plurality of nodes responsible to process reward claims from a second user device associated with the second wearable device. The method may comprise receiving a result of correlation from the second node for the first PA burst data of the first user device with a second PA burst data of the second user device and detecting fraud for disqualification of the reward claim, based on the result being a high correlation of the first PA burst data with the second PA burst data.

Embodiments of the present disclosure may provide a computer program product including at least one non-transitory computer-readable storage medium having computer-executable program code stored therein. The computer program product comprising a non-transitory computer readable medium having stored thereon computer executable instructions, which when executed by a computer, cause the computer to carry out operations, for obtaining first PA data of a first wearable device associated with a first user. The first wearable device may be linked to a first user device registered with the PAAS. The first user may claim for a reward based on the first PA data and detecting whether the first PA data is faulty PA data generated by the first wearable device worn by a non-owner of the first wearable device, based on fraud detection criteria.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Having thus described example embodiments of the disclosure in general terms, reference will now be made to the accompanying drawings, which are not necessarily drawn to scale, and wherein:

FIG. 1 is a block diagram that illustrates an environment for a Physical Activity Assessment System (PAAS) for detecting faulty physical activity (PA) data for claiming a reward, in accordance with an example embodiment;

FIG. 2 illustrates a block diagram of the PAAS, exemplarily illustrated in FIG. 1, that may be used for detecting faulty physical activity (PA) data for claiming a reward, in accordance with an example embodiment;

FIG. 3 illustrates detection of a heart rate of a user (such as the first user) by the first user device reported by its paired first wearable device indicating a start of an intensive exercise, in accordance with an embodiment;

FIGS. 4A and 4B illustrates tabular representation of PA data set and PA burst data set respectively of a wearable device, in accordance with an embodiment;

FIG. 4C illustrates a flowchart for implementation of an exemplary method for fraud detection of PA data, in accordance with an embodiment;

FIG. 4D illustrates a tabular representation of a reward claim format with number of PA bursts in PA data set, in accordance with an embodiment;

FIG. 4E illustrates fraud detection for PA data set by a decentralized application (DApp) on a distributed server (e.g. a blockchain node), in accordance with an embodiment;

FIG. 4F illustrates a flowchart for implementation of an exemplary method for fraud detection based on Nearby Device ID on nodes of the distributed server, in accordance with an embodiment;

FIG. 5A illustrates a tabular representation of reward claim format with family and friend group, in accordance with an embodiment;

FIG. 5B illustrates a tabular representation of GPS PoI set of the first user on the first user device, in accordance with an embodiment;

FIG. 6A illustrates a tabular representation of PA data entry format with GPS values, in accordance with an embodiment;

FIG. 6B illustrates a tabular representation of PA burst data, in accordance with an embodiment;

FIG. 6C illustrates an exemplary scenario for fraud detection for PA data set with GPS location by a decentralized application (DApp) on a distributed server (e.g. a blockchain node), in accordance with an embodiment;

FIG. 6D illustrates a flowchart for implementation of an exemplary method for GPS based fraud detection of PA data, in accordance with an embodiment; and

FIG. 7 illustrates a flowchart for implementation of an exemplary method for detection of faulty physical activity (PA) data for claiming a reward, in accordance with an embodiment.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced item. Moreover, various features are described which may be exhibited by some embodiments and not by others. Similarly, various requirements are described which may be requirements for some embodiments but not for other embodiments. As used herein, the terms “data,” “content,” “information,” and similar terms may be used interchangeably to refer to data capable of being displayed, transmitted, received and/or stored in accordance with embodiments of the present disclosure. Thus, use of any such terms should not be taken to limit the spirit and scope of embodiments of the present disclosure.

As defined herein, a “computer-readable storage medium,” which refers to a non-transitory physical storage medium (for example, volatile or non-volatile memory device), may be differentiated from a “computer-readable transmission medium,” which refers to an electromagnetic signal.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

A system, a method, and a computer program product are provided herein in accordance with an example embodiment to detect faulty physical activity (PA) data for claiming a reward. More particularly, various embodiments of the present disclosure provide a physical activity assessment system (PAAS) to detect faulty physical activity (PA) data presented for claiming a reward, the PAAS may comprise at least one non-transitory memory configured to store computer-executable instructions and at least one processor configured to execute the computer-executable instructions to obtain first PA data of a first wearable device associated with a first user, wherein the first wearable device is linked to a first user device registered with the PAAS, and wherein the first user claims for a reward based on the first PA data, and the at least one processor is configured to detect whether the first PA data is one of faulty PA data generated by the first wearable device worn by a non-owner, or authentic PA data generated by the first wearable device worn by the first user who is owner of the first wearable device, based on fraud detection criteria. It shall be noted that the owner of the first wearable device implies that the user (first user) is registered with the first wearable device, and the non-owner of the first wearable device implies that the user is not registered with the first wearable device. Accordingly, hereinafter, the term owner will be interchangeably used as “registered user” and the term “non-owner” may be interchangeably used as “non-registered user”.

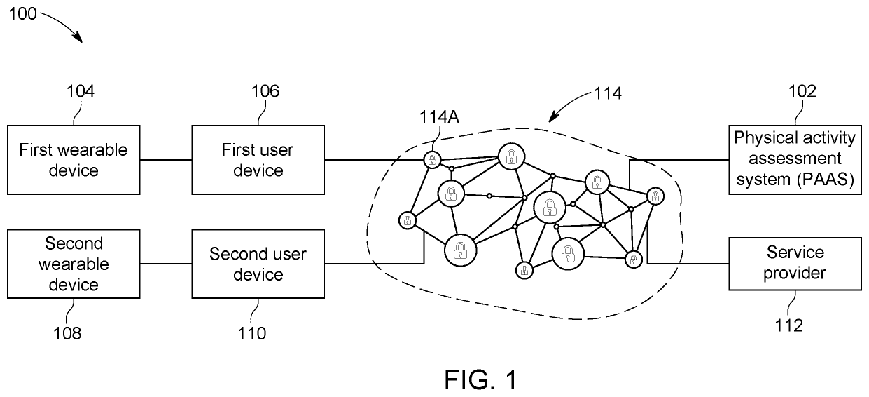

FIG. 1 is a block diagram that illustrates an environment 100 for a Physical Activity Assessment System (PAAS) for detecting faulty physical activity (PA) data for claiming a reward, in accordance with an example embodiment. There is shown an environment 100 that may include a Physical Activity Assessment System (PAAS) 102, a first wearable device 104, a first user device 106, a second wearable device 108, a second user device 110, a service provider 112, and a network 114. The PAAS 102 may be communicatively coupled to the first user device 106 and the second user device 110, via the network 114. The network 114 may be a blockchain network, hereinafter referred as “blockchain network 114”. The PAAS 102 may be communicatively coupled to the service provider 112, via the blockchain network 114. The first wearable device 104 is paired with the first user device 106. Further, the second wearable device 108 is paired with the second user device 110. In accordance with an embodiment, the PAAS 102 may be directly coupled to the service provider 112.

In some example embodiments, the PAAS 102 may be implemented in a cloud computing environment. All the components in the environment 100 may be coupled directly or indirectly to the blockchain network 114. The components described in the environment 100 may be further broken down into more than one component and/or combined together in any suitable arrangement. Further, one or more components may be rearranged, changed, added, and/or removed.

The PAAS 102 may comprise suitable logic, circuitry, and interfaces that may be configured to detect faulty physical activity (PA) data presented for claiming a reward. The PAAS 102 may be configured to obtain first PA data of the first wearable device 104 associated with a first user (not shown in the FIG. 1). The first wearable device 104 may be linked to the first user device 106 which may be registered with the PAAS 102. The first user claims for the reward based on the first PA data generated by the first wearable device 104 during a physical activity. The PAAS 102 may be configured to detect whether the first PA data is one of faulty PA data generated by the first wearable device 104 worn by a non-owner (i.e. worn by someone other than the first user), or authentic PA data generated by the first wearable device 104 worn by the first user who is owner of the first wearable device 104. In an example embodiment, the PAAS 102 may be a server, group of servers, distributed computing system, and/or other computing system.

The first wearable device 104 may comprise suitable logic, circuitry, hardware components and interfaces that may be configured to generate the first PA data, and provide the first PA data to the first user device 106 associated with the first user. The first wearable device 104 may be paired with the first user device 106. The PA data (the first PA data and the second PA data) may comprise, but not limited to, heart rate data, pulse rate, quality of sleep, or walking steps data. The heart rate data comprises a heart rate of a user, wearing at least one of the first wearable device or the second wearable device, for a finite period of time. The heart rate comprises a number of heart beats of the user in one minute. The first wearable device 104 may be linked to only one PAAS account. Examples of the first wearable device 104 may include, but not limited to, fitness bands, smart-watches, fitness rings, activity trackers and fitness neck loops.

The first user device 106 may comprise suitable logic, circuitry, and interfaces that may be configured to receive the first PA data from the first wearable device 104, and provide the first PA data to the PAAS 102 to claim the reward. The first user device 106 may run a PAAS application and may register to the PAAS 102 by the owner (the first user) of the first user device 106.

Each person or user may only be able to register to the PAAS 102 with only one account. Similarly, a second user may be able to register to the PAAS 102 with only one account (different from the account of the other users i.e. the first user) via the second wearable device 108 for the second user device 110.

The service provider 112 may comprise suitable logic, circuitry, hardware components and interfaces that may be configured to provide reward to a claimant, such as the first user for authentic PA data generated by the first wearable device 104. In accordance with an embodiment, the PAAS 102 may be directly coupled to the service provider 112. Examples of the service provider 112 may include, but not limited to, reward sponsors or third party entities.

The blockchain network 114 may comprise suitable logic, circuitry, and interfaces that may be configured to provide a plurality of network ports and a plurality of communication channels for transmission and reception of data, such as the PA data. The blockchain network 114 may have the core characteristics of decentralization, accountability, and security. The blockchain network 114 may have peer-to-peer (P2P) network. The blockchain network 114 may comprise a plurality of nodes, such as a first node 114A. Each node within the blockchain network 114 may be configured to maintain, approve, and update new entries. Each member in the blockchain network 114 may ensure that all records and procedures are in order, which results in data validity and security. Thus, the PAAS 102 and the service provider 112 are able to reach a common consensus. The blockchain network 114 may comprise of many computers, but in a way that the data cannot be altered without the consensus of the whole blockchain network 114 (each separate computer). The structure of blockchain network 114 may be represented by a list of blocks with transactions in a particular order. These lists can be stored as a flat file (txt. format) or in the form of a simple database. Examples of the blockchain network 114 may include, but not limited to, public blockchain network, private blockchain network and consortium blockchain network. Communication data may be transmitted or received, via communication protocols.

In operation, the PAAS 102 may be configured to receive a request from the first user device 106 to claim a reward for the physical activity performed by the first user. The first user may wear the first wearable device 104 and perform physical activities (PA). The first wearable device 104 may be configured to measure the first PA data of the first user and transmits the first PA data to the paired first user device 106. The PA data may include without limitation, a measure of physical activity of a user. For example, the PA data may include distance covered while walking, brisk walking, running, cycling, exercising etc. and time taken in doing so. Additionally, or alternately, the PA data may include change in weight of the user over a period of time, heart rate, breathing rate, pulse rate captured over a period of time. The first user device 106 may accumulate the first PA data of the first user until it reaches a threshold to claim a reward. The PAAS 102 may be configured to authenticate the first PA data generated by the first wearable device 104 with accurate PA values and timestamps. In accordance with an embodiment, the first PA data is authenticated based on determination that errors on the PA values and the timestamps may be within tolerable ranges. A reward executor may be implemented on the blockchain network 114 to generate one or more rewards for claim of the first user, based on a smart contract. Similarly, a second user may wear the second wearable device 108 to claim a reward, via the second user device 110, for a physical activity by presenting the second PA data determined by the second wearable device 108. The service provider 112 may provide funds to the reward executor on the blockchain network 114, based on terms and conditions in the same smart contract or a different smart contract.

The PAAS system may be configured to detect faulty PA data when the first wearable device 104 may be worn by a person (say, the second user) other than the owner (the first user) or the second wearable device 108 may be worn by the first user who is not the owner of the second wearable device 108. In other scenario, one person may wear two or more wearable devices, such as the first user may wear both the first wearable device 104 and the second wearable device 108 at same time during an exercise. The PAAS 102 may implement fraud detection mechanisms on the wearable devices (such as the first wearable device 104), the user devices (such as the first user device) and/or the blockchain nodes (such as the blockchain node 114A).

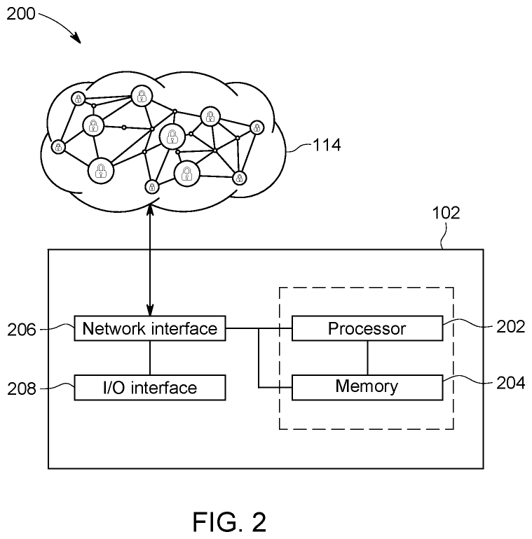

FIG. 2 illustrates a block diagram 200 of the PAAS 102, exemplarily illustrated in FIG. 1 that may be used for detecting faulty physical activity (PA) data for claiming a reward, in accordance with an example embodiment. FIG. 2 is explained in conjunction with FIG. 1.

In the embodiments described herein, the PAAS 102 may include a processing means, such as, at least one processor (hereinafter interchangeably used with processor) 202, a storage means, such as, at least one memory (hereinafter interchangeably used with memory) 204, a communication means, such as, at least one network interface (hereinafter interchangeably used with network interface) 206 and an I/O interface 208. The processor 202 may retrieve computer program instructions that may be stored in the memory 204 for execution of the computer program instructions. In accordance with an embodiment, the processor 202 may be configured to obtain input (such as, the first PA data from the first user device 106), and render output (such as, detect faulty PA data or authentic PA data) to determine the output associated with the input associated with a user (such as the first user) that interacts with the first user device 106.

The processor 202 may be configured to obtain the first PA data from the first user device 106 associated with the first wearable device 104 which is owned by the first user. In accordance with an embodiment, the processor 202 may be further configured to obtain a trained machine learning model and store the trained machine learning model in the memory 204. The machine learning model may be trained on the PA data. In accordance with an embodiment, the processor 202 may be configured to detect whether the first PA data is one of faulty PA data generated by the first wearable device 104 when worn by a non-owner, or authentic PA data generated by the first wearable device 104 when worn by the first user who is owner of the first wearable device, based on fraud detection criteria. In accordance with an embodiment, the processor 202 may be configured to authenticate the PA data (the first PA data or the second PA data) based on the trained machine learning model.

The processor 202 may be embodied in a number of different ways. The processor 202 may be a specialized processor designed specifically for use with the present disclosure. For example, the processor 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 202 may include one or more processing cores configured to perform independently. A multi-core processor may enable multiprocessing within a single physical package. Additionally or alternatively, the processor 202 may include one or more processors configured in tandem via the bus to enable independent execution of instructions, pipelining and/or multithreading. Additionally, or alternatively, the processor 202 may include one or more processors capable of processing large volumes of workloads and operations to provide support for big data analysis. In an example embodiment, the processor 202 may be in communication with the memory 204 via a bus for passing information.

Alternatively, as another example, when the processor 202 is embodied as an executor of software instructions, the instructions may specifically configure the processor 202 to perform the algorithms and/or operations described herein when the instructions are executed. However, in some cases, the processor 202 may be a processor specific device (for example, a fixed computing device) configured to employ an embodiment of the present disclosure by further configuration of the processor 202 by instructions for performing the algorithms and/or operations described herein. The processor 202 may include, among other things, a clock, a timer, an arithmetic logic unit (ALU) and logic gates configured to support operation of the processor 202. The environment, such as, 100 may be accessed using the network interface 206. The network interface 206 may provide an interface for accessing various features and data stored in the PAAS 102.

The memory 204 may be configured to store the first PA data obtained from the first user device 106. In accordance with an embodiment, the memory 204 may be configured to store the machine learning model (not shown in the FIG. 2) that may be trained and tested on the PA data (the first PA data or the second PA data). In accordance with an embodiment, the machine learning model may be trained by a third party service provider and obtained by the processor 202 to be stored in the memory 204. In accordance with an embodiment, the memory 204 may be configured to store software that is to be manipulated by commands to the processor 202. The memory 204 may be configured to store data that has to be transmitted by the PAAS 102 as an output to the first wearable device 106. In accordance with an embodiment, the memory 204 may be configured to store intermediate data used to carry out the steps of the PAAS 102. In accordance with an embodiment, the memory 204 may include storing historical data associated with a user (such as the first user) associated with the first wearable device 104.

The memory 204 may be non-transitory and may include, for example, one or more volatile and/or non-volatile memories. In other words, for example, the memory 204 may be an electronic storage device (for example, a computer readable storage medium) comprising gates configured to store data (for example, bits) that may be retrievable by a machine (for example, a computing device like the processor 202). The memory 204 may be configured to store information, data, content, applications, and instructions for enabling the PAAS 102 to carry out various functions in accordance with an example embodiment of the present disclosure. For example, the memory 204 may be configured to buffer input data for processing by the processor 202. As exemplarily illustrated in FIG. 2, the memory 204 may be configured to store instructions for execution by the processor 202. As such, whether configured by hardware or software methods, or by a combination thereof, the processor 202 may represent an entity (for example, physically embodied in circuitry) capable of performing operations according to an embodiment of the present disclosure while configured accordingly. Thus, for example, when the processor 202 is embodied as an ASIC, FPGA or the like, the processor 202 may be specifically configured hardware for conducting the operations described herein. In accordance with an embodiment, the memory 204 may be accessed using an encryption key for parameters such as the PA data.

The network interface 206 may comprise suitable logic, circuitry, and interfaces that may be configured to communicate with the components of the PAAS 102 and other systems and devices in the environment 100, via the blockchain network 114. The network interface 206 may communicate with the first user device 106, via the blockchain network 114 under the control of the processor 202. The network interface 206 may provide an interface for accessing various data stored in the PAAS 102.

In some example embodiments, the I/O interface 208 may communicate with the first user device 106 and displays input and/or output for the first user device 106. In accordance with an embodiment, the input may correspond to the first PA data of the first wearable device 104 associated with the first user device 106 that may be transmitted to the PAAS 102, via the I/O interface 208. In accordance with an embodiment, the output may correspond to a text message for the first user 110 from the PAAS 102 regarding violation of timestamps associated with the PA data. The output may also be provided to other devices or other programs; e.g., to other software modules, for use therein.

In one embodiment, the PAAS 102 may comprise user interface circuitry configured to control at least some functions of one or more I/O interface elements. The processor 202 may be configured to control one or more functions of one or more I/O interface elements of the I/O interface 208 through computer program instructions (for example, software and/or firmware) stored on the memory 204 accessible to the processor 202.

In some embodiments, the processor 202 may be configured to provide Internet-of-Things (IoT) related capabilities to users of the wearable devices registered with the PAAS 102 disclosed herein. The IoT related capabilities may in turn be used to provide smart solutions by providing real time updates and big data analysis, by using the cloud based system for providing recommendation services.

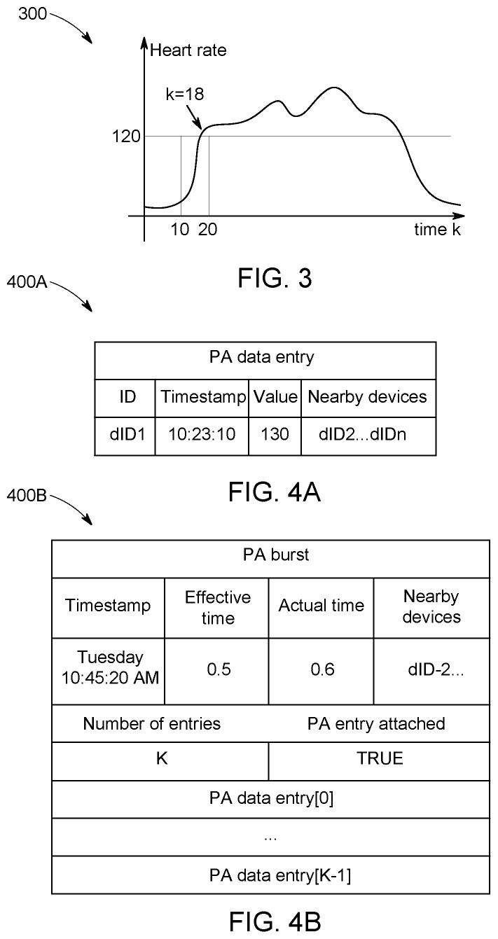

FIG. 3 illustrates a graphical representation of detection of a heart rate of a user (such as the first user) by the first user device 106 reported by its paired first wearable device 104 indicating a start of an intensive exercise. FIG. 3 is explained in conjunction with FIG. 1 and FIG. 2.

With reference to FIG. 3, X axis represents time intervals from 0 to k with a time interval of 10 units such as (0, 10, 20 . . . k), k can be any finite number, for example, but not limited to, 15, 18, 20, 25 or the like, and Y axis represents the heart rate of the first user. Physical activity (PA) data generated by the first wearable device 104 may be referred as the first PA data. The first PA data may comprise a first heart rate for a first time period. To determine correlation of PA data sets (say a first PA data and a second PA data), heart rate as a parameter may be used. The second PA data is generated by the second wearable device 108 paired with the second user device 110.

Therefore, the PAAS 102 may be configured to determine a correlation of the first heart rate in the first PA data of the first wearable device 104 generated for the first time period with a second heart rate of the second PA data of the second wearable device 108 generated for a second time period which overlaps with the first time period. In accordance with an embodiment, the degree of overlap of the second time period with the first time period may be high. The correlation may be a high correlation, based on value of the correlation being greater than a first pre-defined threshold value. The PAAS may be further configured to determine a correlation period, comprising a finite number of time instances, of the correlation between the first PA data and the second PA data. The correlation period corresponds to a time overlap between the first time period and the second time period, and wherein the correlation period.

The PAAS 102 may be further configured to flag the first PA data of the first wearable device 104 and the second PA data of the second wearable device 108 generated for the first time period as the faulty PA data, based on the high correlation.

In an exemplary scenario, heart rate measured by the first wearable device 104 and the second wearable device 108 is {R1[k1]} and {R2[k2]}, respectively, where k refers to the same time period for both the first wearable device 104 and the second wearable device 108. It is assumed that wearable devices cannot tamper their timestamps. The PAAS 102 may be further configured to setup or tune clock of all registered wearable devices, based on a network timer such that all registered wearable devices timers are synchronized.

The cross variance between two PA data sets (the first PA data from the first wearable device 104 and the second PA data from the second wearable device 108) of K samples at K synchronized time instances is calculated by the PAAS 102 as:

$$Cross variance ( {Var }_{1 , 2} [ K ] ) = \frac{Σ k = 1 … K \{ [ R 1 [ k ] - r 1 ] [ R 2 [ k ] - r 2 ] \}}{( Sqrt [ Var 1 ] * Sqrt [ Var 2 ] )}$$

where r1 and r2 are means of {R1[k]} and {R2[k]} for K samples, respectively and where Var1 and Var2 are variances of {R1[k]} and {R2[k]} for K samples, respectively.

When the cross-variance may be greater than a threshold value (for example, but not limited to, 0.9), the two PA data sets may be flagged as fraud. A PAAS application on the first user device 106 and the second user device 110 may decide to prompt an alarm for first violation of terms and conditions of the PAAS application, and disqualify the first PA data for reward claim if it happens frequently.

Alternative ways to determine the correlation between two PA data sets may include using first and/or second derivatives of the PA data (for example, the first derivative of the first PA data and the second derivative of the second PA data) which can filter the differences on noise and bias of different devices. The computing complexity may be reduced as well.

In an example embodiment, the PAAS may further be configured to determine presence of missing data (i.e. whether heart beats are missing in the heart rate data) in the {R1[k]} and {R2[k]} for K samples during a finite time duration. Upon determination, the PAAS may further be configured to fill the missing data with an average value (for example, but not limited to, an average of heart rate during the k time period) using at least one of an interpolation technique or an extrapolation technique. The occurrence of the missing data in the heart rate data may indicate one of an over-exertion of the user due to involvement in a physical activity, an illness of the user, or the like.

With reference to the graphical representation of FIG. 3, the first user device 106 may be configured to detect the heart rate reported by its paired first wearable device 104 and may be more than a threshold value (say 120 for k=18-20), indicating an intensive exercise start. The first user device 106 may start to record a PA burst from k=18 to k=20 until the heart rate is below 120. For better fraud detection, a user device (such as the first user device 106) may be required to provide an extended PA burst for an exercise, including at least one of a rest time, a ramp up period, an exercise period, a slow-down period, and a recovery period. Correlation of the extended PA bursts from two devices (the first user device 106 and the second user device 110) may be more accurate in determining when the two wearable devices (say, the first wearable device 104 and the second wearable device 108) are worn by the same person.

A person may use a trick by taking on and off one of the wearable devices to reduce the correlation of two PA bursts data sets. A PA burst data with sudden change during an exercise period may raise the attention by the PAAS 102 for fraud detection. The sudden change indicates a presence of the missing data in the first PA burst data during an extended time period of the physical activity. The missing data may occur due to palpitation. For better fraud detection performance, the PAAS 102 may require the users to wear their wearables devices before and after the exercise and warn them that otherwise the PA data may be disqualified to claim a reward.

In accordance with an embodiment, a wearable device (such as the first wearable device 104) may be configured to collect biometric information of owner (the first user) in many ways. The PAAS 102 may require a user (such as, the first user) to wear the wearable device (such as the first wearable device 104) in the extended exercise period. The PAAS application on the paired user device (such as, the first user device 106) may be able to analyze the PA data set to see when rest time heart rate, ramp up and slow down patterns, the recovery time and heart rate pattern are significantly different from historical data collected from the owner (the first user). The historical data collected from the owner (the first user) may correspond to the biometric information of owner (the first user). The PA data generated by the exercise of non-owner will be disqualified for reward claim.

In general, the non-owner detection may use machine learning model to identify a PA data set belonging to an owner based on large volume of the historical data. The personal identification based on PA data may be hard among a large group of people. Of a small group that a user may be able to share his wearable device, the probability of finding another person with the similar feature set is very low.

In accordance with an embodiment, the PAAS 102 may be configured to use the second derivatives of PA data set to identify the owner. Generally, the heart rate acceleration from rest to exercise and from exercise to recovery may be very typical for an individual. When the second derivatives of a PA data set may offset from the historical pattern significantly, a non-owner may be detected.

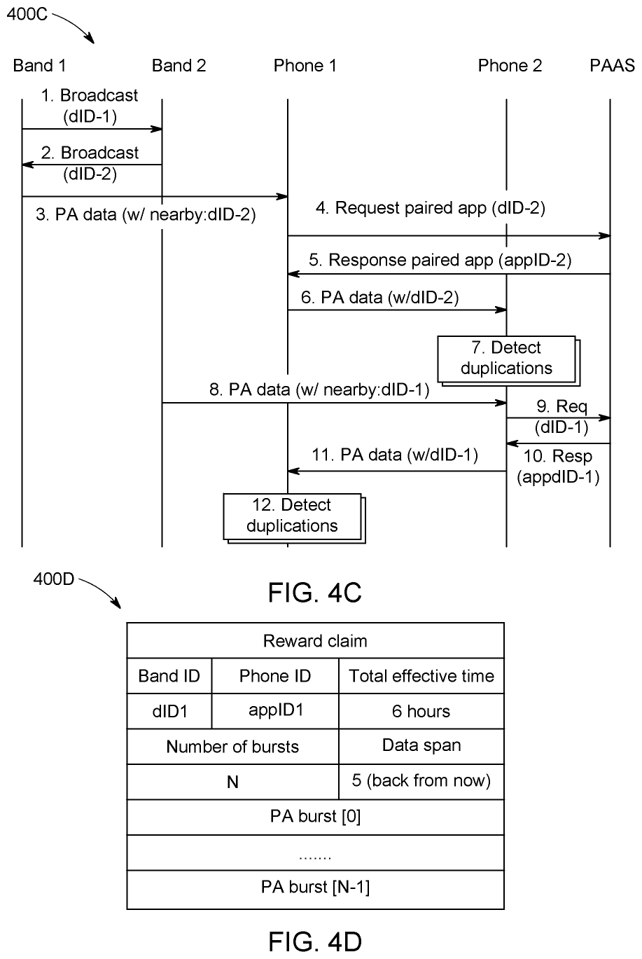

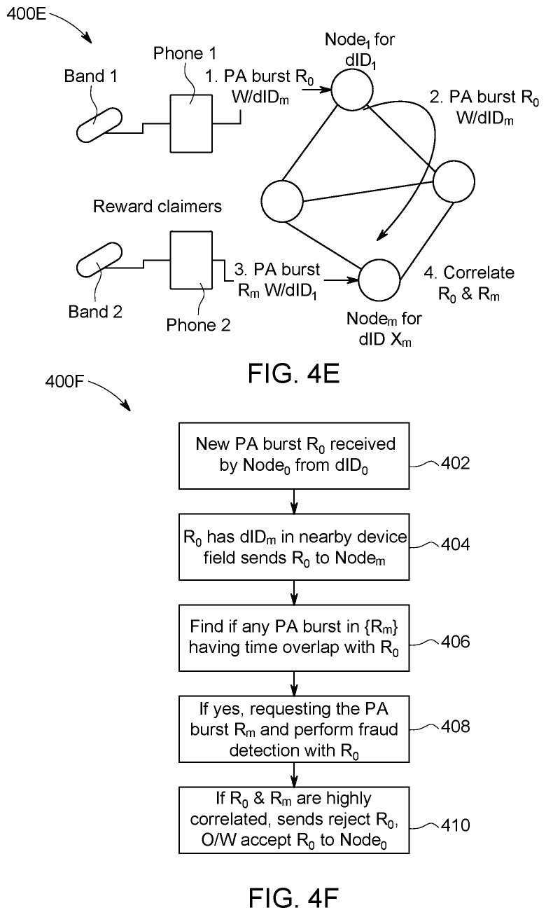

FIGS. 4A and 4B illustrate tabular representation of PA data set and PA burst data set of a wearable device, in accordance with an embodiment. FIGS. 4A and 4B are explained in conjunction with FIG. 1 to FIG. 3.

A person may register multiple accounts with multiple wearable devices such that the PAAS 102 may fail to detect the owner of a wearable device. However, the PAAS 102 may be configured to detect two PA burst data that are highly correlated to detect the fraud, especially between family members and/or friend groups.

The PAAS 102 may be configured to use existing beacon message to piggyback PA data whenever the PA data reaches a certain threshold value. Such use of the existing beacon message may require no peer-to-peer (P2P) connectivity between two wearable devices (such as the first wearable device 104 and the second wearable device 108), which is a low cost solution as compared to changing firmware. Since the wearable devices (such as, the first wearable device 104) are broadcasting and listening to the beacons to/from other wearable devices (such as, the second wearable device 108) anyway, the solution requires no hardware modification. The only requirement is to modify the format of beacon and define a protocol to exchange the PA data. Such solution may be standardized on PA data exchange protocol, when wearable devices (such as, the first wearable device 104 and the second wearable device 108) are from different vendors. In addition, privacy is an issue to broadcast the PA data in clear text. Encryption may require group session key establishment and distribution that may further complicate the PAAS 102. An alternative solution based on existing beacon messages may be that a wearable device (such as the first wearable device 104) broadcasts beacon messages periodically. In accordance with an embodiment, the frequency of beacon messages broadcast may be increased when the PA value in the PA data set (such as, the first PA data) reaches a threshold value (for example, heart rate is equal to 120). The broadcast signal may remain at low power to reduce interference. A nearby wearable device (such as, the second wearable device 108) may receive the beacon message with identity (dID) of the first wearable device. When the second wearable device 108 continuously receives the beacon messages of the first wearable device 104, the second wearable device 108 may add the identity (dID) of the first wearable device 104 in a nearby device field in its own PA data entry until it no longer receives the beacon messages of the first wearable device 104.

With reference to the FIG. 4A, the PA data format may be shown. Each PA data entry has a source ID, a timestamp, a value and a nearby device field of the first wearable device 104 that may be transmitted to the paired user device (the first user device 106) in real-time or synchronized in bulk. The first user device 106 may correspond to a smart phone.

With reference to the FIG. 4B, the user device (such as, the first user device 106) may receive the first PA data from its paired wearable device (such as, first wearable device 104) and packs it into a PA burst data with a format shown. A PA burst data may comprise data associated with a physical activity during an exercise period that may count towards a reward. The nearby device field may comprise only the identities (dIDs) received consistently during the exercise. Such setup may rule out use cases, such as, two persons are nearby each other for a short period on the road or they start or end the exercises at different times in a gym.

FIG. 4C illustrates a flowchart for implementation of an exemplary method for fraud detection of PA data, in accordance with an embodiment. FIG. 4C is explained in conjunction with FIG. 1 to FIG. 4B.

There is shown a band 1, a band 2, a phone 1, a phone 2 and the PAAS 102. The band 1 may correspond to the first wearable device 104. The band 2 may correspond to the second wearable device 108. The phone 1 may correspond to the first user device 106. The phone 2 may correspond to the second user device 110. At first and second step, the first wearable device 104 and the second wearable device 108 may broadcast beacon messages with their identities (IDs). One embodiment is the first wearable device 104 and the second wearable device 108 may be configured to broadcast beacon messages at a higher frequency than the default when their respective PA data values in the first PA data and the second PA data may reach a threshold value.

At third step, the first user device 106 may be configured to receive PA burst data from the first wearable device 104 with dID-2 in nearby device field.

At fourth step, the first user device 106 may be configured to transmit a request to find the paired app ID-2 of dID-2 from the PAAS 102. Since each wearable device (the first wearable device 104 and the second wearable device 108) is paired to only one phone application and account, the appID-2 may be identified.

At fifth step, the PAAS 102 may be configured to return the appID-2 of the second wearable device 108 registered with the second user device 110.

At sixth step, the first user device 106 may be configured to transmit fraud detection request with the PA burst that includes PA data entries with dID-2 to the application on the second user device 110 for fraud detection.

At seventh step, the second user device 110 may be configured to detect if there is a PA burst data received from the second wearable device 108 with high correlation to the first PA data set from the first wearable device 104. If detected, the application on the second user device 110 may drop the PA burst data that correlates to the first PA burst data from the first user device 106 or the second user device 110 may transmit a fraud detection response to the first user device 106 indicating a fraud is detected, then the first user device 106 may drop the PA burst data.

At eighth to twelfth steps, the same process for the PA burst data with PA data having dID-1 in nearby device field from the second wearable device 108, if the second user device 110 has not yet received a fraud detection request from the first user device 106 and processed the PA burst.

The application on the user device (such as the first user device 106) may accumulates the PA burst data from its paired wearable device (such as the first wearable device 104) until the total effective PA time equals or exceed a threshold value (for example, 5 hours).

In some embodiments, certain ones of the operations above may be modified or further amplified. Furthermore, in some embodiments, additional optional operations may be included. Modifications, additions, or amplifications to the operations above may be performed in any order and in any combination.

FIG. 4D illustrates a tabular representation of a reward claim format with number of PA bursts in PA data set, in accordance with an embodiment. FIG. 4D is explained in conjunction with FIG. 1 to FIG. 4C.

The application on a user device may generate a reward claim with number of PA bursts as illustrated in FIG. 4D. Since user devices, (the first user device 106 and the second user device 110) may not be able to exchange PA bursts data when they are not connected to the network at the same time or they are on different private networks, the reward claim may be delayed significantly. Band ID which corresponds to the first wearable device ID is shown. Further, phone ID for the first user device, total effective time, number of bursts, dates span is shown in the FIG. 4D.

FIG. 4E illustrates fraud detection for PA data set by a decentralized application (DApp) on a distributed server (e.g. a blockchain node), in accordance with an embodiment. FIG. 4E is explained in conjunction with FIG. 1 to FIG. 4D.

A distributed server may be assigned to process the reward claims from given set of dIDs/appIDs for load balance purpose. As shown in FIG. 4E, the first user device 106 (or a Phone1) paired with the first wearable device 104 (or Band1) with dID1 may transmit a reward claim with a PA burst R0 to Node1 which may be responsible to process claims from dID1. The DApp on the Node1 may process the claim and finds a PA burst R0 having dID m in its nearby device field and transmit a fraud detection request message to Node m which may be responsible to process reward claims from dID m. The Node m may perform the correlation check when the PA burst R0 may be detected fraud due to high correlation with a PA burst Rm, one or both bursts may be disqualified to claim the reward.

FIG. 4F illustrates a flowchart for implementation of an exemplary method for fraud detection based on Nearby Device ID on nodes of the distributed server, in accordance with an embodiment. FIG. 4F is explained in conjunction with FIG. 1 to FIG. 4E.

Firstly, a new PA burst R0 having a timestamp (t0, T0) may be received 402 at Node 0 from device with dID 0, which is one of the PA burst from its reward claim. Secondly, the R0 comprises 404 a nearby device field with dID m. The DApp on Node 0 may transmit R0 to Node m which is responsible to process claims from dID m. There could be multiple {Nodem} in the blockchain responsible to process claims of dID m. Thirdly, Node m may search 406 for any PA burst Rm from {Rm} that has enough time overlap with RO. Fourthly, when Rm is found, PA data of burst Rm may be requested 408 and fraud detection with PA data of the burst R0 may be performed. Finally, when R0 and Rm may be highly correlated 410, Node m may transmit a rejection on R0 to Node 0, otherwise, Node m may transmit an acceptance. The DApp may decide to reject the whole reward claim when one PA burst in the claim may be detected fraud, or only deduct the PA burst from a claim and reduce the amount of reward accordingly.

In some embodiments, certain ones of the operations above may be modified or further amplified. Furthermore, in some embodiments, additional optional operations may be included. Modifications, additions, or amplifications to the operations above may be performed in any order and in any combination.

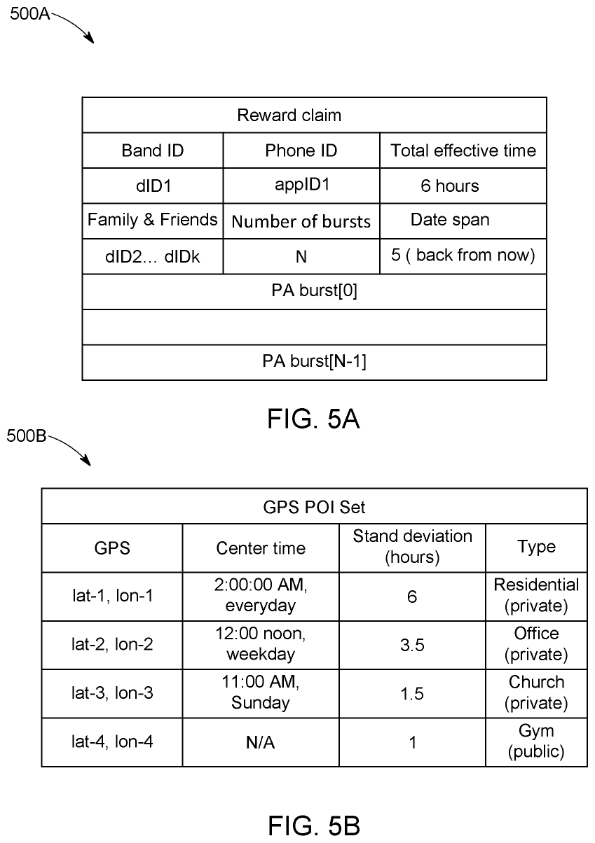

FIG. 5A illustrates a tabular representation of reward claim format with family and friend group, in accordance with an embodiment. FIG. 5A is explained in conjunction with FIG. 1 to FIG. 4F.

The requirement on wearable devices to broadcast device ID during the exercise period may have challenges on product compatibility among multiple vendors that may also prevent the PAAS 102 to use off the shelf wearable devices. Since it may be unlikely that a person wears a device of a stranger and claim reward for that person using his exercises, the duplicated PA burst submissions may likely happen among people who know each other.

The application on the first user device 106 may know the identities of its owner's (the first user's) family and friends. The PAAS 102 may request a reward claim which includes the family and friend's dIDs (or applDs) in a family & friends field. The same fraud detection procedure in FIG. 4E may apply except the nearby device set is replaced by a family and friend's device set. It may be assumed that the likelihood of a fraud requiring collaboration of unrelated persons is low.

FIG. 5B illustrates a tabular representation of GPS PoI set of the first user on the first user device, in accordance with an embodiment. FIG. 5B is explained in conjunction with FIG. 1 to FIG. 5A.

A person may register multiple wearable devices via multiple phones on multiple accounts but intentionally may keep accounts unrelated, that is, the persons may not explicitly friend each other. To address this potential fraud, the PAAS 102 may be configured to use GPS point of interest (PoI) to build a virtual friend group (VFG) for each user account. A PoI may be a location that a user stays for significant period regularly, such as a gym, an office or a home. The PoI may be classified into a public PoI and private PoI. The public PoI may be a location where unrelated people go, such as a gym, and a mall. When two users stay in the private PoI, such as home or office, the two users may be likely known to each other. Search space for fraud detection by the PAAS 102 may be targeted to a group of people who have known each other.

The reward application on the first user device (that may be a smartphone) may transmit GPS PoI set of the first user, as shown in FIG. 5B, to the PAAS 102 and update it regularly. This information is normally available in account of users for most of wearable products. The PAAS 102 may be configured to create a virtual friend group (VFG) for the first user, based on one or more rules. For example, the second user may have at least one non-public PoI in common with the first user. Another rule may be that the number of users in the PoI may be less than K, (say 10 people). PoI with too many users, such as a large corporation, may not be used. Further, the second user may have a residential PoI in common with the first user and they may likely be the family members. Further, large apartment complex building may be excluded.

Furthermore, a second user may have two or more non-public Pols in common with the second user and that may likely be the family members or close friends. Then, a second user may have one non-public PoI in common and one exercise PoI in common with the first user. An exercise PoI may correspond to a location where PA bursts may be often generated.

The PoI may use network address (e.g. IP address) in addition to the GPS value, that is, the second user with the same public IP address as that of the first user may be identified in the same PoI.

The PAAS 102 may make a tradeoff between the size of VFG and inclusive of the true family and friends in the group. For a person who may register multiple accounts with multiple wearable devices may have the home as the common PoI. The PAAS 102 may be configured to put weights on different type of Pols, for example, home/residential Pols have more weight than office Pols.

The PAAS 102 may be configured to use the dIDs (or appIDs) to identify the friends in the VFG. When a user device (such as, the first user device 106) may submits a reward claim to the PAAS 102, the first user device 106 may add the dIDs (or appIDs) of the VFG in the family & friends field in the PA burst data. Again, the method of FIG. 4F may apply, except the nearby device field may be replaced by a VFG field. Since the public PoI may not be used for VFG setup, only private POIs may be considered. The PAAS 102 may use third party tool to determine a location is a public PoI or a private PoI.

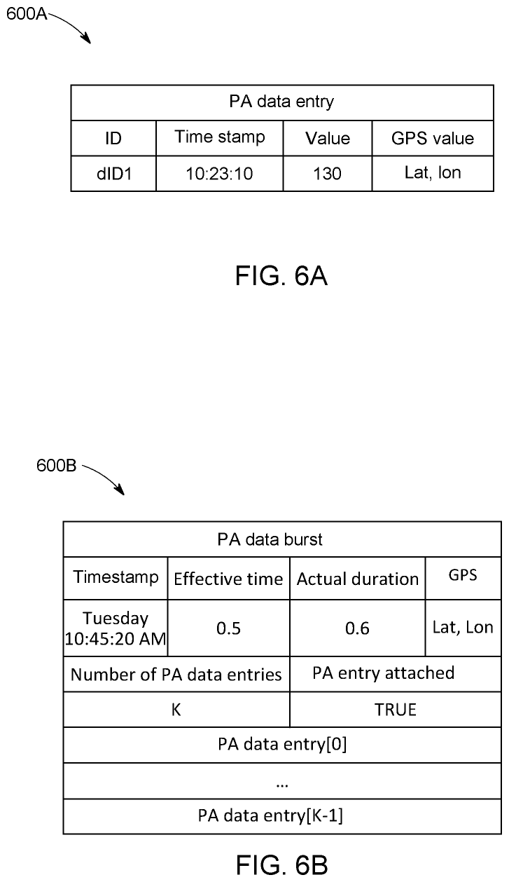

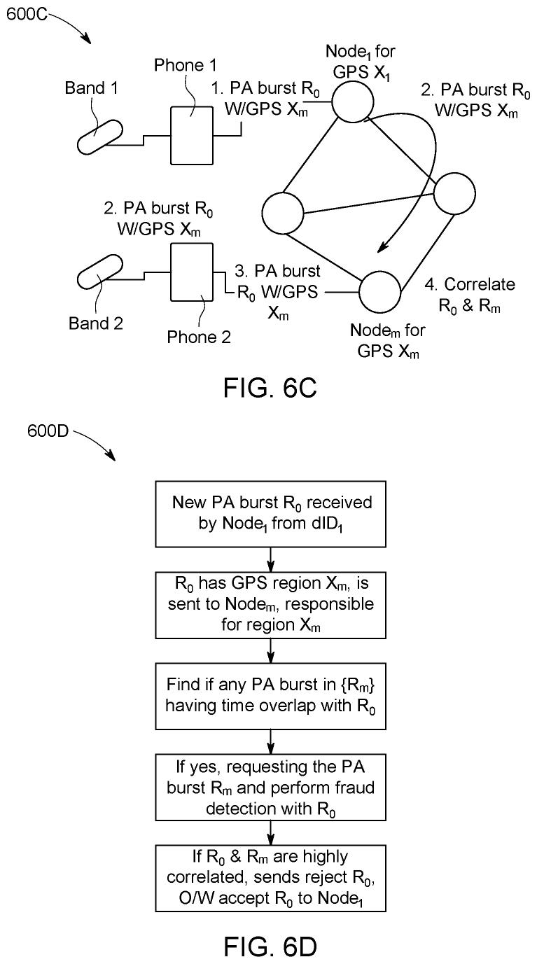

FIG. 6A illustrates a tabular representation of PA data entry format with GPS values, in accordance with an embodiment. FIG. 6A is explained in conjunction with FIG. 1 to FIG. 5B.

The PAAS 102 may be configured to detect faulty PA data based on GPS value of a place where a user exercises. Commonly, most of the wearable devices for sports and recreation purposes embed a GPS to trace the location of the user. The application on the paired user device may get the GPS trace whenever they are in-sync. With the time correlation, each PA data entry can have a GPS value stored in the GPS field, as shown in FIG. 6A.

FIG. 6B illustrates a tabular representation of PA burst data, in accordance with an embodiment. FIG. 6B is explained in conjunction with FIG. 1 to FIG. 6A.

The burst from the PA burst data may also have a GPS field, representing the location or trace of the place of exercise of the user. The PA burst data may optionally have all PA data entries, and each may have a GPS value. For an indoor exercise, the GPS signal may be unavailable, and then the PA data entry may use nearest GPS available before the PA burst data may start. In accordance with an embodiment, the PAAS 102 may use the GPS coordinates provided by map service for indoor facilities, such as a home and an office building.

FIG. 6C illustrates an exemplary scenario for fraud detection for PA data set with GPS location by a decentralized application (DApp) on a distributed server (e.g. a blockchain node), in accordance with an embodiment. FIG. 6C is explained in conjunction with FIG. 1 to FIG. 6B.

A distributed server may have a plurality of nodes, (e.g. blockchain nodes) that may be assigned to process the reward claims based on their GPS values on their PA bursts. For example, a claim may include five different GPS values of their PA bursts, the claim may be processed by up to five nodes. The assignment may be based on zip code, that is, when the GPS of a PA burst may fall in a zip code area responsible by a node A, the PA burst data may be transmitted to the node A for antifraud detection. One distributed node may cover multiple zip code areas.

With reference to FIG. 6C, there is shown a band 1, band 2, phone 1, phone 2, a plurality of nodes from node 1 to node m. The band 1 and the band 2 may correspond to wearable devices. The phone 1 and phone 2 may correspond to the user devices registered with the distributed server for claiming rewards based on generation of PA data from wearable devices. The phone1 may submit a reward claim. The reward claim may include PA burst R0 with a GPS location Xm to node 1. The node 1 may be responsible to process PA bursts in areas including GPS location X1. The PA burst may be forwarded to the node m which is responsible to process PA bursts in coverage areas including GPS Xm. At the node m, when R0 may have time overlap with one of {Rm}, cross-correlation of R0 and Rm may be computed.

On the distributed server node, the DApp may detect when a newly received PA burst may be highly correlated to any of past PA bursts which are spatially and temporally close to it. When a newly received PA burst may be highly correlated to any of past PA bursts, the PA burst may be considered fraud detection.

FIG. 6D illustrates a flowchart for implementation of an exemplary method for GPS based fraud detection of PA data, in accordance with an embodiment. FIG. 6D is explained in conjunction with FIG. 1 to FIG. 6C.

A new PA burst R0 may have a timestamp (t0, T0) that may be received at Nodel from the first user device 106 with dID1, which is one of the PA burst from its reward claim. The R0 may comprise a GPS field with value Xm, for a location region Xm. The DApp on Nodel may transmit RO to Node m which may be responsible to process claims for GPS region Xm. There may be multiple nodes { Nodem} in the blockchain that may be responsible to process GPS region Xm claims. Node m may search for any PA burst Rm from {Rm} that may have enough time overlap with R0. When Rm may be found, requesting the PA data of burst Rm and performing fraud detection with PA data of the burst R0. When R0 and Rm may be highly correlated, Node m may transmit a rejection on R0 to Nodel, otherwise, Node m may transmit an acceptance.

The DApp at a distributed node may process a PA burst, when the distributed node finds a past PA burst is spatial and temporal close enough to the new PA burst. Further, a correlation check may be performed. When the past PA burst and the new PA burst are highly correlated, the newly received the PA burst may be considered a fraud.

The method 600D may be performed only for those PA bursts that cannot pass the owner test. A PA burst may be labeled with confident level of owner generated. For example, a PA burst with complete extended data and fit to historical pattern of the owner may be labeled with a higher confident level. When the band (wearable device) may be registered by a user providing his personal identity, such as social security number (required by insurance company), the PA burst may be labeled with even higher confident level.

In accordance with an embodiment, the PAAS 102 may be configured to randomly select the PA data associated with a user. The fraud detection from the generated PA data may be a costly procedure for the distributed servers (e.g. a blockchain network), the DApp may perform the procedure at a low probability, e.g., 1%, that is, only request the PA burst correlation once of every hundred pair of PA bursts from the PA burst data which meet criteria of potential fraudulent. A user that may frequently make fraud reward claims may be caught at a higher probability. Once the user may be caught with a fraudulent, subsequent penalty may be applied to the user.

After a reward claim passed the fraud detection, one or more reward transactions may be created under the consensus of a set of blockchain nodes which create a block that contains the corresponding transactions.

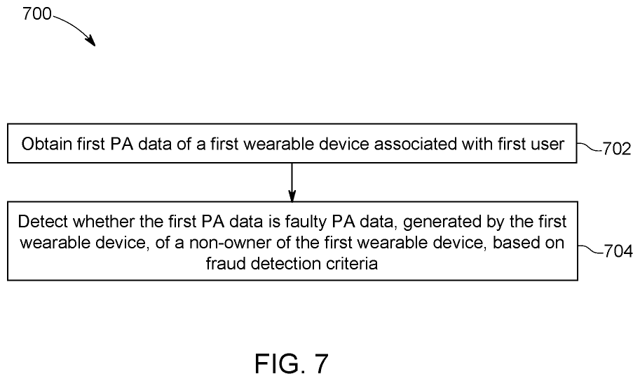

FIG. 7 illustrates a flowchart 700 for implementation of an exemplary method for detection of faulty physical activity (PA) data for deceitful claiming of a reward, in accordance with an embodiment. FIG. 7 is explained in conjunction with FIG. 1 to FIG. 6D.

At 702, first PA data of a first wearable device associated with a first user may be obtained. The processor 202 of the PAAS 102 may be configured to obtain first PA data of a first wearable device associated with a first user. The first wearable device may be linked to a first user device registered with the PAAS. The first user may claim for a reward based on the first PA data.

At 704, it may be detected whether the first PA data is faulty PA data generated by the first wearable device worn by a non-owner of the first wearable device. The processor 202 of the PAAS 102 may be configured to detect whether the first PA data is faulty PA data generated by the first wearable device worn by a non-owner of the first wearable device, based on fraud detection criteria. A first criterion that a second user who is the non-owner of the first wearable device wears the first wearable device associated with the first user for a single physical activity or a second criterion that the first user wears the first wearable device and a second wearable device for the single physical activity, wherein the second wearable device is not registered by the first user.

In some embodiments, certain ones of the operations above may be modified or further amplified. Furthermore, in some embodiments, additional optional operations may be included. Modifications, additions, or amplifications to the operations above may be performed in any order and in any combination.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

We claim:

1. A physical activity assessment system (PAAS) to detect faulty physical activity (PA) data for claiming a reward, the PAAS comprising:
at least one non-transitory memory configured to store computer-executable instructions; and
at least one processor configured to execute the computer-executable instructions to:
obtain first PA data of a first wearable device associated with a first user, wherein the first wearable device is linked to a first user device registered with the PAAS, and wherein the first user claims for a reward based on the first PA data; and
detect whether the first PA data is faulty PA data generated by the first wearable device of a non-owner of the first wearable device, based on fraud detection criteria.

2. The PAAS of claim 1, wherein the fraud detection criteria comprise one or more of:
a first criterion that a second user who is the non-owner of the first wearable device wears the first wearable device associated with the first user for a single physical activity; or
a second criterion that the first user wears the first wearable device and a second wearable device for the single physical activity, wherein the second wearable device is not registered by the first user.

3. The PAAS of claim 2, further comprising a network timer, wherein the at least one processor is further configured to synchronize a time stamp of a plurality of wearable devices that are registered with the PAAS based on the network timer, and wherein the plurality of wearable devices that are registered with the PAAS comprises the first wearable device and the second wearable device.

4. The PAAS of claim 3,
wherein the first PA data comprises first PA burst data including a first heart rate for a first time period, and first extended PA burst data,
wherein second PA data, generated from the second wearable device for a second time period, comprises second PA burst data and second extended PA burst data,
wherein the second PA burst data comprises a second heart rate generated at the second time period which partially overlaps with the first time period, and
wherein the at least one processor is further configured to:
determine a correlation of the first heart rate in the first PA data of the first wearable device with the second heart rate in the second PA data, based on the first PA burst data, the first extended PA burst data, the second PA burst data and the second extended PA burst data,
wherein the correlation is a high correlation based on value of the correlation being greater than a first pre-defined threshold value; and
flag at least one of the first PA data of the first wearable device or the second PA data of the second wearable device generated for the first time period as the faulty PA data, based on the high correlation,
wherein the high correlation associates with the first criterion or the second criterion.

5. The PAAS of claim 4, wherein a correlation period of the correlation between the first PA data and the second PA data corresponds to a time overlap between the first time period and the second time period, and wherein the correlation period comprises a finite number of time instances.

6. The PAAS of claim 4, wherein the at least one processor is further configured to calculate a cross variance between the first PA data of the first wearable device and the second PA data for determination of the correlation of the first PA data of the first wearable device and the second PA data of the second wearable device.

7. The PAAS of claim 6, wherein the cross variance is calculated as:
$$cross variance ( {Var }_{1 , 2} [ K ] ) = \frac{Σ k = 1 … K \{ [ R 1 [ k ] - r 1 ] [ R 2 [ k ] - r 2 ] \}}{( Sqrt [ Var 1 ] * Sqrt [ Var 2 ] )}$$
wherein (Var_{1,2}[K]) is a cross variance between two PA data sets (the first PA data and the second PA data) of K samples at K synchronized time instances,
wherein r1 and r2 are means of {R1[k]} and {R2[k]} for K samples respectively, and
wherein Va1 and Var2 are variances of {R1[]} and {R2[k]} for K samples, respectively.

8. The PAAS of claim 7, wherein the at least one processor is further configured to:
determine missing data in one of the R1[k] or the R2[k] in the K time instances, and
fill the missing data by using at least one of an interpolation technique or an extrapolation technique.

9. The PAAS of claim 4, wherein the at least one processor is further configured to control an application on the first wearable device associated with the first PA data and an application on the second wearable device associated with the second PA data to prompt an alarm for violation based on generation of the faulty PA data.

10. The PAAS of claim 4, wherein the at least one processor is further configured to:
determine first and second derivatives of the first PA data of the first wearable device and the second PA data of the second wearable device; and
determine the high correlation between the first PA data and the second PA data, based on the first and second derivatives of the first PA data and the second PA data respectively.

11. The PAAS of claim 4,wherein the first extended PA burst data and the second extended PA burst data comprises at least one of rest time data, ramp up period data, exercise period data, slow down period data, or recovery period data, and wherein the first PA burst comprises data associated with a physical activity during an exercise period that count towards the claim for reward.

12. The PAAS of claim 11,
wherein the at least one processor is further configured to control an application on the first user device to raise an alarm for fraud detection, based on a sudden change in the first PA burst data during physical activity period, and
wherein the sudden change indicates a presence of the missing data in the first PA burst data during an extended time period of the physical activity.

13. The PAAS of claim 4,
wherein the first PA data of the first wearable device associated with the first user further comprises historical data of the first user who is the owner of the first wearable device, and
wherein the at least one processor is further configured to:
obtain a trained machine learning model trained on the historical data of the first user;
and
detect whether the first PA data is the faulty PA data generated by the first wearable device worn by the non-owner of the first wearable device, based on the trained machine learning model.

14. The PAAS of claim 13, wherein the historical data of the first user corresponds to biometric data of the first user, and wherein the biometric information of the first user comprises at least one of rest time data, heart rate data, ramp up pattern data, slow down pattern data, or recovery time data.

15. The PAAS of claim 14, wherein the at least one processor is further configured to:
determine second derivative of the first PA data; and
detect the first PA data being a faulty PA data generated by the first wearable device worn by a non-owner, based on deviation of the second derivative of the first PA data from the historical data of the first PA data.

16. The PAAS of claim 15, wherein the at least one processor is further configured to:
receive a request from the application of the first user device to find a paired application of the second user device, based on the first user device receiving the first PA burst data from the first wearable device with the second wearable device in a vicinity of the first wearable device's signal coverage;
identify the paired application of the second user device associated with the second wearable device, based on the first wearable device being in a vicinity of the second wearable device;
transmit an application identity number of the second user device to the first user device; and
control the first user device to transmit fraud detection request to the second user device along with the first PA burst data and the application identity number of the second user device, wherein the second user device is in the vicinity of the first user device.

17. The PAAS of claim 11,
wherein the first PA burst data further comprises identity of family and friends of the first user associated with the first wearable device, and
wherein the at least one processor is further configured to detect whether the first PA burst data is faulty PA burst data generated by the first wearable device worn by the non-owner, based on Virtual Friend Group (VFG) criteria.

18. The PAAS of claim 17, wherein the VFG criteria comprises at least one of:
a first criterion that the second user has a common non-public Point of Interest (PoI) with the first user, wherein the non-public PoI comprises home and office, and wherein the home is assigned more weightage than the office as non-public PoI;
a second criterion that a number of people in the non- public PoI is less than a third pre-defined threshold value;
a third criterion that the second user has a common non-public PoI and an exercise PoI with the first user, wherein the exercise PoI corresponds to a location where the first PA burst data is generated; or
a fourth criterion that a network address and a Global Positioning System (GPS) value of the second user is same as that of the first user.

19. A fraud detection server comprising a plurality of nodes, wherein a first node of the plurality of nodes is configured to:
receive a reward claim with a first physical activity (PA) burst data from a first user device paired with a first wearable device associated with a first user, wherein the first PA burst data comprises an identity of a second wearable device in nearby device field having time overlap with the first PA burst data;
transmit a fraud detection request message to a second node of the plurality of nodes responsible to process reward claims from a second user device associated with the second wearable device;
receive a result of correlation, from the second node, for the first PA burst data of the first user device with a second PA burst data of the second user device; and
detect fraud for disqualification of the reward claim, based on the result being a high correlation of the first PA burst data with the second PA burst data.

20. The fraud detection server of claim 18, wherein the first node of is further configured to accept qualification of the reward claim of the first user device, based on the result being a non-correlation of the first PA burst data with the second PA burst data.

21. The fraud detection distributed server of claim 18, wherein the first node is responsible for processing reward claim from the first user device and wherein the first user device is registered with the fraud detection distributed server.

22. The fraud detection distributed server of claim 18, wherein the plurality of nodes correspond to blockchain nodes.

23. A method for detecting faulty physical activity (PA) data for claiming a reward, the method comprising:
obtaining first PA data of a first wearable device associated with a first user, wherein the first wearable device is linked to a first user device registered with the PAAS, and wherein the first user claims for a reward based on the first PA data; and
detecting whether the first PA data is one of faulty PA data generated by the first wearable device of a non-owner the first wearable device, based on fraud detection criteria.24. A computer program product comprising a non-transitory computer readable medium having stored thereon computer executable instructions, which when executed by one or more processors, cause the one or more processors to carry out operations for detecting faulty physical activity (PA) data for claiming a reward, the operations comprising:
obtaining first PA data of a first wearable device associated with a first user, wherein the first wearable device is linked to a first user device registered with the PAAS, and wherein the first user claims for a reward based on the first PA data; and
detecting whether the first PA data is one of faulty PA data generated by the first wearable device of a non-owner of the first wearable device, based on fraud detection criteria.
